(19) United States
(12) Patent Application Publication
(10) Pub. No.: US 2005/0059661 A1
(43) Pub. Date: Mar. 17, 2005

(54) Use of tyrosine kinase inhibitors for the treatment of inflammatory processes

(75) Inventors: Birgit Jung, Laupheim (DE); Hubert Pueschner, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/899,817

(22) Filed: Jul. 27, 2004

Related U.S. Application Data
(60) Provisional application No. 60/495,540, filed on Aug. 15, 2003.

(30) Foreign Application Priority Data
Jul. 28, 2003 (DE) DE 103 34 226.5

(51) Int. Cl. A61K 31/5377

(52) U.S. Cl. 514/234.2

(57) ABSTRACT

The present invention relates to the use of selected quinazolines, the tautomers, stereoisomers and salts thereof, particularly the physiologically acceptable salts thereof with inorganic or organic acids or bases, for preparing a pharmaceutical composition for the prevention or treatment of diseases of the airways or lungs as well as other inflammatory diseases.

### RELATED APPLICATIONS

This application claims priority benefit of U.S. Application Ser. No. 60/495,540, filed Aug. 15, 2003 and DE 10334226.5 filed Jul. 28, 2003 each of which is hereby incorporated by reference in its entirety.

### TECHNICAL FIELD OF THE INVENTION

Quinazolines compounds have been reported in the literature for treating diseases, especially tumor diseases, disorders of the lung and of the respiratory tract, WO 02/18373, WO 02/50043, WO 01/77104, WO 02/18351, WO 02/18372, WO 03/082290.

The present invention relates to the use of quinazolines selected from the group consisting of
(1) 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline,
(2) 4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline,
(3) 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
(4) 4-[(3-ethynyl-phenyl)amino]-6-{[4-(5,5-dimethyl-2-oxo-morpholin-4-yl)-1-oxo-2-buten-1-yl]amino}-quinazoline,
(5) 4-[(3-chloro-4-fluoro-phenyl)amino]-6-[2-(2,2-dimethyl-6-oxo-morpholin-4-yl)-ethoxy]-7-methoxy-quinazoline,
(6) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(7) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(8) 4-[(3-chloro-4-fluoro-phenyl)amino]-6-{2-[4-(2-oxo-morpholin-4-yl)-piperidin-1-yl]-ethoxy}-7-methoxy-quinazoline,
(9) 4-[(3-chloro-4-fluoro-phenyl)amino]-6-[cis-4-(N-methanesulphonyl-N-methyl-amino)-cyclohexan-1-yloxy]-7-methoxy-quinazoline,
(10) 4-[(3-chloro-4-fluoro-phenyl)amino]-6-[cis-4-(N-acetyl-N-methyl-amino)-cyclohexan-1-yloxy]-7-methoxy-quinazoline,
(11) 4-[(3-chloro-4-fluoro-phenyl)amino]-6-(trans-4-methylamino-cyclohexan-1-yloxy)-7-methoxy-quinazoline,
(12) 4-[(3-chloro-4-fluoro-phenyl)amino]-6-[trans-4-(N-methanesulphonyl-N-methyl-amino)-cyclohexan-1-yloxy]-7-methoxy-quinazoline,
(13) 4-[(3-chloro-4-fluoro-phenyl)amino]-6-(trans-4-dimethylamino-cyclohexan-1-yloxy)-7-methoxy-quinazoline,
(14) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(15) 4-[(3-chloro-4-fluoro-phenyl)amino]-6-{1-[2-(2-oxo-3-methyl-imidazolidin-1-yl)ethyl]-piperidin-4-yloxy}-7-methoxy-quinazoline,
(16) 4-[(3-chloro-4-fluoro-phenyl)amino]-6-{1-[2-(2-oxo-hexahydropyrimidin-1-yl)ethyl]-piperidin-4-yloxy}-7-methoxy-quinazoline,
(17) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(18) 4-[(3-chloro-4-fluoro-phenyl)amino]-6-(1-methanesulphonyl-piperidin-4-yloxy)-7-methoxy-quinazoline and
(19) 4-[(3-chloro-4-fluoro-phenyl)amino]-6-(1-cyano-piperidin-4-yloxy)-7-methoxy-quinazoline,
   the tautomers, stereoisomers and salts thereof, particularly the physiologically acceptable salts thereof with inorganic or organic acids or bases, for preparing a pharmaceutical composition for the prevention and treatment of diseases of the airways and lungs which are accompanied by increased or altered production of mucus, e.g. in inflammatory diseases of the airways such as acute bronchitis, chronic bronchitis, chronic obstructive bronchitis (COPD), asthma, bronchiectasias, allergic or non-allergic rhinitis or sinusitis, cystic fibrosis, α1-antitrypsin deficiency, or coughs, pulmonary emphysema, pulmonary fibrosis or hyperreactive airways.

The compounds are also suitable for treating inflammatory diseases of the gastrointestinal tract or bile duct and gall bladder which are associated with disrupted activity of the tyrosine kinases, such as may be found e.g. in acute or chronic inflammatory changes such as cholecystitis, Crohn's disease, ulcerative colitis, and ulcers or polyposis in the gastrointestinal tract or such as may occur in diseases of the gastrointestinal tract which are associated with increased secretions, such as Ménétrier's disease, secreting adenomas and protein loss syndromes,

and also for treating inflammatory diseases of the joints, such as rheumatoid arthritis, inflammatory diseases of the skin, the eyes, in inflammatory pseudopolyps, in colitis cystica profunda or pneumatosis cystoides intestinalis.

Preferred fields of application are inflammatory diseases of the respiratory organs or of the intestine, such as chronic bronchitis (COPD), chronic sinusitis, asthma, Crohn's disease, ulcerative colitis or polyposis of the intestines.

Particularly preferred fields of application are inflammatory diseases of the airways or lungs such as chronic bronchitis (COPD) or asthma.

The present invention further relates to a process for treating

diseases of the airways and lungs which are accompanied by increased or altered production of mucus, e.g. in inflammatory diseases of the airways such as acute bronchitis, chronic bronchitis, chronic obstructive bronchitis (COPD), asthma, bronchiectasias, allergic or non-allergic rhinitis or sinusitis, cystic fibrosis, α1-antitrypsin deficiency, or coughs, pulmonary emphysema, pulmonary fibrosis and hyperreactive airways,

for treating inflammatory diseases of the gastrointestinal tract and bile duct and gall bladder which are associated with disrupted activity of the tyrosine kinases, such as may be found e.g. in acute or chronic inflammatory changes such as cholecystitis, Crohn's disease, ulcerative colitis and ulcers or polyposis in the gastrointestinal tract or such as may occur in diseases of the gastrointestinal tract which are associated with increased secretions, such as Ménétrier's disease, secreting adenomas and protein loss syndromes,

and also for treating inflammatory diseases of the joints, such as rheumatoid arthritis, inflammatory diseases of the skin, the eyes, in inflammatory pseudopolyps, in colitis cystica profunda and pneumatosis cystoides intestinalis,

comprising administering an effective amount of one or more of the above-mentioned compounds (1) to (19) or optionally one of the physiologically acceptable salts thereof to a patient requiring such treatment.

In the process according to the invention the above-mentioned compounds are used in doses from 0.001-10 mg/kg body weight, for example 0.5-7.0 mg/kg, preferably 0.01-1.5 mg/kg, expediently administered 1 to 3 times a day.

The active substances may be administered by oral, buccal or parenteral route, by atomisation for inhalation, rectally or topically. They may be administered parenterally by subcutaneous, intravenous and intramuscular injections and infusion techniques.

Conventional formulations may be used for administering them, such as the formulations mentioned above with regard to the active substances. For example, the active substances may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetyl stearyl alcohol, carboxymethylcellulose or fatty substances such as hard fat or suitable mixtures thereof, to produce conventional galenic preparations such as plain or coated tablets, capsules, powders, suspensions or suppositories.

The active substances may be given orally in a variety of different dosage forms, i.e. they may be prepared with various pharmaceutically acceptable inert carriers to form tablets, capsules, pastilles, lozenges, sweets, powders, sprays, aqueous suspensions, elixirs, syrups and the like. Such carriers include solid diluents or fillers, sterile aqueous media and various non-toxic organic solvents. In addition, oral pharmaceutical preparations of this kind may be suitably sweetened or flavoured, using various agents conventionally used for this purpose. In general the active substances are present in oral formulations of this kind in concentrations ranging from about 0.5 to about 90 wt. %, based on the total composition, in amounts sufficient to produce the desired dosage units. Other suitable dosage forms for the active substances include preparations and devices with controlled release, with which the skilled man will be familiar.

For parenteral administration solutions of the active substances in sesame or groundnut oil or in aqueous propyleneglycol as well as sterile aqueous solutions of the corresponding pharmaceutically acceptable salts may be used. Aqueous solutions of this kind should be suitably buffered as necessary and the liquid diluent may optionally be made isotonic with sufficient salt or glucose. These specific aqueous solutions are particularly suitable for intravenous, intramuscular and subcutaneous injection. In this context, the sterile aqueous media used may easily be obtained by conventional methods well known to the skilled man. For example, distilled water is normally used as a liquid diluent, and the finished preparation is passed through a suitable bacterial filter, e.g. a filter made of sintered glass, kieselguhr or unglazed porcelain. Preferred filters of this type include Berkefeld, Chamberland and asbestos disc metal Seitz filters, the liquid being aspirated into a sterile container using a suction pump. Throughout the entire process of preparing these injectable solutions the necessary steps should always be carried out in such a way as to obtain the end products in a sterile state. For transdermal administration the formulations of the particular compounds or compounds include for example solutions, lotions, ointments, creams, gels, suppositories, formulations for long-lasting speed-limited release preparations and devices therefor. These formulations comprise the particular compound(s) and may contain ethanol, water, penetration promoters and inert carriers, e.g. gel-forming materials, mineral oil, emulsifiers, benzyl alcohol and the like.

The substances may be administered by inhalation in the form of powder formulations with lactose and other excipients or in the form of aqueous solutions as an aerosol.

The inhalable powders which may be used according to the invention may contain the active substance or combination of active substances either on its own or in admixture with suitable physiologically acceptable excipients. If the active substance or combination of active substances is present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextran), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Inhalation aerosols containing propellant gas which may be used according to the invention may contain the active substance or combination of active substances dissolved in the propellant gas or in dispersed form. The propellant gases which may be used to prepare the inhalation aerosols according to the invention are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane) and mixtures thereof.

The propellant-driven inhalation aerosols which may be used according to the invention may also contain other ingredients such as co-solvents, stabilisers, surface-active agents (surfactants), antioxidants, lubricants and pH adjusters. All these ingredients are known in the art.

If the active substance or combination of active substances according to the invention is administered by inhalation in the form of propellant-free solutions or suspension, aqueous or alcoholic, preferably ethanolic solutions may be used as the solvent. The solvent may be exclusively water or a mixture of water and ethanol. The relative proportion of ethanol to water is not restricted, but the maximum limit is up to 70 percent by volume, more particularly up to 60 percent by volume and most preferably up to 30 percent by volume. The remainder of the volume is made up of water. The solutions or suspensions containing the active substance or combination of active substances are optionally adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric acid and sulphuric acid. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

As already mentioned hereinbefore, the compounds of general formula (I) and the salts thereof have valuable properties, particularly an anti-inflammatory activity.

For example, the compounds

A=4-[(R)-(1-phenyl-ethyl)amino]-6-{[4-((R)-6-methyl-2-oxo-morpholin-4-yl)-1-oxo-2-buten-1-yl]amino}-7-methoxy-quinazoline,
B=4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
C=4-[(3-chloro-4-fluoro-phenyl)amino]-6-[2-(2,2-dimethyl-6-oxo-morpholin-4-yl)-ethoxy]-7-methoxy-quinazoline,
D=4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline and
E=4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline
   were tested as follows to investigate their anti-inflammatory activity:
   Test 1: Inhibition of the Smoke-Induced Accumulation of Granulocytes in the Lung Tissue

Lung indications: Inhibition of the cigarette smoke-induced influx of neutrophilic granulocytes into the lung tissue by the EGF-receptor kinase inhibitor 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline (compound A).

Method:

Male rats (strain: Sprague-Dawley) weighing from 250-300 g were exposed for 5 days to the smoke from 8 cigarettes per day. The animals in the group treated with compound A, anaesthetised with isofluran, were given an intratracheal dose of 0.03 or 0.1 mg/kg of compound A in a volume of 0.05 ml each day 30 min before the start of the exposure to smoke. On the last day of the test the animals were killed 4 hours after the last exposure to smoke and the lung tissue was removed. A sample of 70-200 mg was taken from each lung and placed in a test tube prepared with 1 ml of 0.5% hexadecyltrimethylammonium bromide. The samples were homogenised for 15 sec using an Ultraturrax. The homogenised samples were centrifuged at 15700 g in an Eppendorf bench centrifuge for 5 min at ambient temperature. 50 ml of the supernatant was removed and mixed with 250 ml of phosphate buffer (50 mmol/l) containing 0.197 mg/ml of O-dianisidine dihydrochloride. After 10 minutes' incubation at ambient temperature the absorption was measured with a spectral photometer at a wavelength of 450 nm.

The dosage which led to a 50% inhibition of the MPO activity (═ID50) was determined by linear regression.

Results:

Exposure to cigarette smoke in rats led to an influx of neutrophilic granulocytes into the lung tissue, measured by the content of myeloperoxidase in the tissues, which is specific for neutrophilic granulocytes. Intratracheal treatment of the animals with the EGFR kinase inhibitor A resulted in a significant (p<0.005) inhibition of the smoke-induced accumulation of granulocytes and thus produced an anti-inflammatory activity.

Further results are shown in the following Table:

| active substance | ID50 [mg/kg] |
|---|---|
| A | 0.3 |
| B | 0.2 |
| C | 1 |
| D | 1.1 |
| E | 0.4 |

1. A method of treatment or prevention of diseases of:
the airways and lungs which are accompanied by increased or altered production of mucus, inflammatory diseases of the gastrointestinal tract or bile duct or gall bladder which are associated with disrupted activity of the tyrosine kinases, inflammatory diseases of the joints, inflammatory diseases of the skin, the eyes, inflammatory pseudopolyps, colitis cystica profunda or pneumatosis cystoides intestinalis,
comprising administration of an effective amount to a person in need of such treatment of a quinazline selected from the group of:
(1) 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline,
(2) 4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazol ine,
(3) 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
(4) 4-[(3-ethynyl-phenyl)amino]-6-{[4-(5,5-dimethyl-2-oxo-morpholin-4-yl)-1-oxo-2-buten-1-yl]amino}-quinazoline,
(5) 4-[(3-chloro-4-fluoro-phenyl)amino]-6-[2-(2,2-dimethyl-6-oxo-morpholin-4-yl)-ethoxy]-7-methoxy-quinazoline,
(6) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(7) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(8) 4-[(3-chloro-4-fluoro-phenyl)amino]-6-{2-[4-(2-oxo-morpholin-4-yl)-piperidin-1-yl]-ethoxy}-7-methoxy-quinazoline,
(9) 4-[(3-chloro-4-fluoro-phenyl)amino]-6-[cis-4-(N-methanesulphonyl-N-methyl-amino)-cyclohexan-1-yloxy]-7-methoxy-quinazoline,
(10) 4-[(3-chloro-4-fluoro-phenyl)amino]-6-[cis-4-(N-acetyl-N-methyl-amino)-cyclohexan-1-yloxy]-7-methoxy-quinazoline,
(11) 4-[(3-chloro-4-fluoro-phenyl)amino]-6-(trans-4-methylamino-cyclohexan-1-yloxy)-7-methoxy-quinazoline,
(12) 4-[(3-chloro-4-fluoro-phenyl)amino]-6-[trans-4-(N-methanesulphonyl-N-methyl-amino)-cyclohexan-1-yloxy]-7-methoxy-quinazoline,
(13) 4-[(3-chloro-4-fluoro-phenyl)amino]-6-(trans-4-dimethylamino-cyclohexan-1-yloxy)-7-methoxy-quinazoline,
(14) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(15) 4-[(3-chloro-4-fluoro-phenyl)amino]-6-{1-[2-(2-oxo-3-methyl-imidazolidin-1-yl)ethyl]-piperidin-4-yloxy}-7-methoxy-quinazoline,
(16) 4-[(3-chloro-4-fluoro-phenyl)amino]-6-{1-[2-(2-oxo-hexahydropyrimidin-1-yl)ethyl]-piperidin-4-yloxy}-7-methoxy-quinazoline,
(17) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(18) 4-[(3-chloro-4-fluoro-phenyl)amino]-6-(1-methanesulphonyl-piperidin-4-yloxy)-7-methoxy-quinazoline and
(19) 4-[(3-chloro-4-fluoro-phenyl)amino]-6-(1-cyano-piperidin-4-yloxy)-7-methoxy-quinazoline,
or the tautomers, stereoisomers and salts thereof, particularly the physiologically acceptable salts thereof with inorganic or organic acids or bases.

2. The method of claim 1 for the treatment of the upper and lower respiratory organs or intestines.

3. The method according to claim 2, wherein the diseases are COPD, chronic sinusitis, asthma, cystic fibrosis, Crohn's disease, ulcerative colitis or polyposis of the intestine.

4. The method of claim 3, wherein the diseases are COPD, asthma or cystic fibrosis.

5. Method of treating diseases of the airways and lungs which are accompanied by increased or altered production of mucus chosen from,
inflammatory diseases of the gastrointestinal tract or bile duct or gall bladder which are associated with disrupted activity of the tyrosine kinases,
inflammatory diseases of the joints, inflammatory diseases of the skin, the eyes, inflammatory pseudopolyps, colitis cystica profunda or pneumatosis cystoides intestinalis, said method comprising administering an effective amount of a quinazoline selected from the group consisting of
(1) 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline,
(2) 4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline,
(3) 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
(4) 4-[(3-ethynyl-phenyl)amino]-6-{[4-(5,5-dimethyl-2-oxo-morpholin-4-yl)-1-oxo-2-buten-1-yl]amino}-quinazoline,
(5) 4-[(3-chloro-4-fluoro-phenyl)amino]-6-[2-(2,2-dimethyl-6-oxo-morpholin-4-yl)-ethoxy]-7-methoxy-quinazoline,
(6) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(7) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(8) 4-[(3-chloro-4-fluoro-phenyl)amino]-6-{2-[4-(2-oxo-morpholin-4-yl)-piperidin-1-yl]-ethoxy}-7-methoxy-quinazoline,
(9) 4-[(3-chloro-4-fluoro-phenyl)amino]-6-[cis-4-(N-methanesulphonyl-N-methyl-amino)-cyclohexan-1-yloxy]-7-methoxy-quinazol ine,
(10) 4-[(3-chloro-4-fluoro-phenyl)amino]-6-[cis-4-(N-acetyl-N-methyl-amino)-cyclohexan-1-yloxy]-7-methoxy-quinazoline,
(11) 4-[(3-chloro-4-fluoro-phenyl)amino]-6-(trans-4-methylamino-cyclohexan-1-yloxy)-7-methoxy-quinazoline,
(12) 4-[(3-chloro-4-fluoro-phenyl)amino]-6-[trans-4-(N-methanesulphonyl-N-methyl-amino)-cyclohexan-1-yloxy]-7-methoxy-quinazoline,
(13) 4-[(3-chloro-4-fluoro-phenyl)amino]-6-(trans-4-dimethylamino-cyclohexan-1-yloxy)-7-methoxy-quinazoline,
(14) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(15) 4-[(3-chloro-4-fluoro-phenyl)amino]-6-{1-[2-(2-oxo-3-methyl-imidazol idin-1-yl)ethyl]-piperidin-4-yloxy}-7-methoxy-quinazoline,
(16) 4-[(3-chloro-4-fluoro-phenyl)amino]-6-{1-[2-(2-oxo-hexahydropyrimidin-1-yl)ethyl]-piperidin-4-yloxy}-7-methoxy-quinazoline,
(17) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(18) 4-[(3-chloro-4-fluoro-phenyl)amino]-6-(1-methanesulphonyl-piperidin-4-yloxy)-7-methoxy-quinazoline and
(19) 4-[(3-chloro-4-fluoro-phenyl)amino]-6-(1-cyano-piperidin-4-yloxy)-7-methoxy-quinazoline,
the tautomers, stereoisomers and salts thereof, particularly the physiologically acceptable salts thereof with inorganic or organic acids or bases, to a patient requiring such treatment.

6. Method according to claim 5 for the treatment of the upper and lower respiratory organs or of the intestines.

7. Method according to claim 6 for the treatment of diseases selected from the list consisting of COPD, chronic sinusitis, asthma, cystic fibrosis, Crohn's disease, ulcerative colitis or polyposis of the intestine.

8. Method according to claim 7, wherein the diseases are selected from the list consisting of COPD, asthma or cystic fibrosis.
